(19) United States
(12) Patent Application Publication
(10) Pub. No.: US 2020/0124691 A1
(43) Pub. Date: Apr. 23, 2020

(54) METHOD TO MODIFY IMAGING PROTOCOLS IN REAL TIME THROUGH IMPLEMENTATION OF ARTIFICIAL INTELLIGENCE

(71) Applicants: David Douglas, Winter Park, FL (US); Kathleen Douglas, Winter Park, FL (US); Robert Douglas, Winter Park, FL (US)

(72) Inventors: David Douglas, Winter Park, FL (US); Kathleen Douglas, Winter Park, FL (US); Robert Douglas, Winter Park, FL (US)

(21) Appl. No.: 16/654,047

(22) Filed: Oct. 16, 2019

Related U.S. Application Data
(60) Provisional application No. 62/852,424, filed on May 24, 2019.
(60) Provisional application No. 62/748,555, filed on Oct. 22, 2018.

(51) Int. Cl. G01R 33/54; G06T 7/00; G06N 20/00; A61B 6/00

(52) U.S. Cl. CPC G01R 33/54; G06T 7/0012; G06T 2207/20081; A61B 6/5205; G06T 2207/10088; G06N 20/00

(57) ABSTRACT

Imaging protocols are modified in real time through implementation of artificial intelligence. Key steps include: inputting of medical images and patient data; performing artificial intelligence analysis; outputting a potentially modified radiological imaging examination protocol; if applicable, an option for the radiologist to review the patient data, images acquired and the AI's potentially modified radiological imaging examination protocol; delivery of the modified radiological imaging examination protocol to the imaging device; and, if applicable, an option for the radiologist to provide feedback and re-train the artificial intelligence algorithm. Further, some of the images that are utilized by the AI system include processed images, such as segmented and filtered volume rendered images.

### TECHNICAL FIELD

Aspects of this disclosure are generally related to use of machines, and more particularly machine learning, in radiology.

### INTRODUCTION

In the field of radiology, but more particularly to MRI scanning, there are commonly inadequate sequences submitted for review. This significantly impairs the radiologist's ability to diagnose certain abnormalities. Due to the typical workflow for radiologists, they do not interrupt their work and stop reviewing the case currently at hand and look at newly acquired images—they get to these new images in accordance with priority and policy of sequencing. As a result, any re-imaging due to inadequacy of the current set of images must be re-scheduled and the patient brought in at some later time.

### SUMMARY

All examples, aspects and features mentioned in this document can be combined in any technically conceivable way.

Aspects of the present disclosure include a method and process to determine if the current image sequence is adequate (or not) using near real time artificial intelligence while the patient is still at the radiological imaging room. If the image sequence is found to be inadequate, a repeated sequence and/or a newly determined sequence(s) would be provided for additional screening which would be performed at that time. This revised protocol will speed the overall process in the radiological unit, save total medical costs, and provide better medical care.

In accordance with an aspect a method comprises: inputting of medical images and patient data; performing artificial intelligence analysis; outputting a potentially modified radiological imaging examination protocol; if applicable, an option for the radiologist to review the patient data, images acquired and the AI's potentially modified radiological imaging examination protocol; delivery of the modified radiological imaging examination protocol to the imaging device; and, if applicable, an option for the radiologist to provide feedback and re-train the artificial intelligence algorithm.

In some embodiments, a whole body MRI could be performed for purposes such as annual screening. The spatial resolution of such a scan could be low, such as 3 mm isotropic. Whole body analysis could be performed. Then, the AI could perform an assessment of the whole-body MRI while the patient is still on the MRI scanner. If the AI doesn't detect an abnormality, then no additional sequences would be required. If desired, the volume could be sent to a database. If the AI did detect an abnormality, then the AI could communicate to the MRI scanner which sequences to perform. The MRI scanner would then perform those sequences. Then, the AI would review those additional sequences. This process would repeat until no additional sequences were deemed necessary by the AI algorithm. Finally, the radiologist would review the MRI examination with the additional AI directed sequences.

In some embodiments, the inputted medical images include one or more of magnetic resonance imaging (MRI), computed tomography (CT), single photon emission computed tomography (SPECT) and positron emission tomography (PET).

In some embodiments, the inputted medical data comprises terminology derived from patient history, terminology derived from radiological examinations, terminology derived from physical examination findings, inputted medical data comprises laboratory data.

In some embodiments, the inputted data would be obtained from feedback from image analysis of patient's anatomical data and patient's posture during the imaging sequence. The resulting data could be relative to the imaging system structure and could be a factor for the AI algorithm. The analysis could include but, would not be limited to: creating a 3D volumetric data set from the 2D image slices; applying segmentation and filtering to volumetric data to determine inter alia position of organs bone and spine structure and cant of the head; and providing these data to the AI algorithm(s). With this data, key aspects of the images could be determined which could include, but are not limited to: angular measurements bone structure, head cant, and position of organs with respect to image boundaries, etc. Metrics could be applied, dependent on the purpose of the imaging session, to determine if additional images were required.

In some embodiments, the inputted data would be obtained from feedback from image analysis of the quality of the image to include but, are not limited to smear within the image. A measure of artifacts (e.g., motion artifact, smear, beam hardening artifact, streak artifact, metallic blooming artifact, chemical shift artifact, aliasing artifact, susceptibility artifact, etc.) would be analyzed through AI training. For example, an AI algorithm could be trained by using 1000 images with susceptibility artifact and 1000 images without susceptibility artifact and the AI algorithm could “learn” how to predict which images have susceptibility artifact and which images do not have susceptibility artifact. Then, the AI algorithm could direct the MRI scanner to perform new sequences designed specifically to minimize susceptibility artifact. Additionally, in the event of motion artifact, which can appear as a smear on an image could be identified by segmentation and filtering of data to determine, for example, sharpness of edges along bone structure. Metrics could be applied, dependent on the purpose of the imaging session, to determine if additional images were required.

In some embodiments, performing analysis comprises utilization of artificial intelligence algorithms, such as convolutional neural networks, recurrent neural networks and reinforcement learning.

In some embodiments, a combination of above methods and processes and sequence of activities may be performed. For example, a patient may have back pain and have a MRI of the back. The physical examination may record the spinal location of the pain in the back. The MRI is taken and then analyzed per wherein, due to the curvature of the spine, an angle of the vertebrae's is computed with respect to the horizontal element of the MRI system. The 3D data set which has been segmented and filtered is then re-oriented to angles (previously X, Y, Z) to new angles X′, Y′, and Z′ such that, viewing the vertebrae from one of these new angles the viewing angle would be perpendicular to a line projected through the vertebrae experiencing pain. (Note: a perpendicular viewing angle increases the probability finding of hard to detect hairline fractures.) This segmented, filtered, and re-oriented volumetric data would then be passed to the artificial intelligence algorithms for further review. Dependent on AI algorithm outcome, the case could be referred to the AI body of knowledge for enhanced reinforcement learning.

In some embodiments, performing analysis comprises assessment of the patient and determination whether repeating the imaging examination is worthwhile.

In some embodiments, performing analysis comprises assessment for pathology in the image(s).

In some embodiments, outputting a modified radiological examination comprises adding new images to be performed (e.g., new MRI sequence).

In some embodiments, the outputted modified radiological examination may be reviewed by the radiologist to accept or reject the modifications to the radiological examination.

In some embodiments, the outputting a modified radiological examination comprises altering the subsequent images scheduled to be performed (e.g., the last sequence of the MRI brain protocol is an axial T2-weighted sequence, and this is changed to a CUBE T2-weighted sequence).

In some embodiments, delivering the modified radiological imaging examination protocol the imaging device can be through human entering modified protocol on the radiological scanner equipment.

In some embodiments, delivering the modified radiological imaging examination protocol to the imaging device can be through an interface between the AI algorithm and the radiological scanner equipment.

In some embodiments, a human reviews the modified radiological imaging examination protocol an adds the dataset comprising of the inputted data and outputted data to a training dataset for machine learning.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 illustrates a flow diagram for the method.

FIG. 2 illustrates inputted medical images includes magnetic resonance imaging (MRI), computed tomography (CT), single photon emission computed tomography (SPECT) and positron emission tomography (PET).

FIG. 3 illustrates examples of inputted medical data.

FIG. 4 illustrates sample artificial intelligence algorithms including deep artificial neural networks and other machine learning algorithms.

FIG. 5 illustrates performing analysis wherein the quality of the image(s) is assessed.

FIG. 6 illustrates assessment of the patient and determination whether repeating the imaging examination is worthwhile.

FIG. 7 illustrates AI altering the subsequent images scheduled to be performed.

FIG. 8 illustrates AI performing analysis of pathology within the images, making recommendations for a modified radiological imaging examination protocol, which is then reviewed by a radiologist and the AI algorithm is subsequently retrained.

FIG. 9 illustrates methods to deliver the modified radiological examination protocol to the scanner.

FIG. 10 illustrates a flow diagram for whole body MRI screening with AI driving sequences.

FIG. 11 illustrates a deep learning algorithm performed on source images to detect cerebral aneurysms, which has been performed in the prior art.

FIG. 12 illustrates a deep learning algorithm performed on processed images to detect cerebral aneurysms.

### DETAILED DESCRIPTIONS

The flow diagrams do not depict the syntax of any particular programming language. Rather, the flow diagrams illustrate the functional information one of ordinary skill in the art requires to fabricate circuits or to generate computer software to perform the processing required in accordance with the present invention. It should be noted that many routine program elements, such as initialization of loops and variables and the use of temporary variables, are not shown. It will be appreciated by those of ordinary skill in the art that unless otherwise indicated herein, the particular sequence of steps described is illustrative only and can be varied without departing from the spirit of the invention. Thus, unless otherwise stated the steps described below are unordered meaning that, when possible, the steps can be performed in any convenient or desirable order.

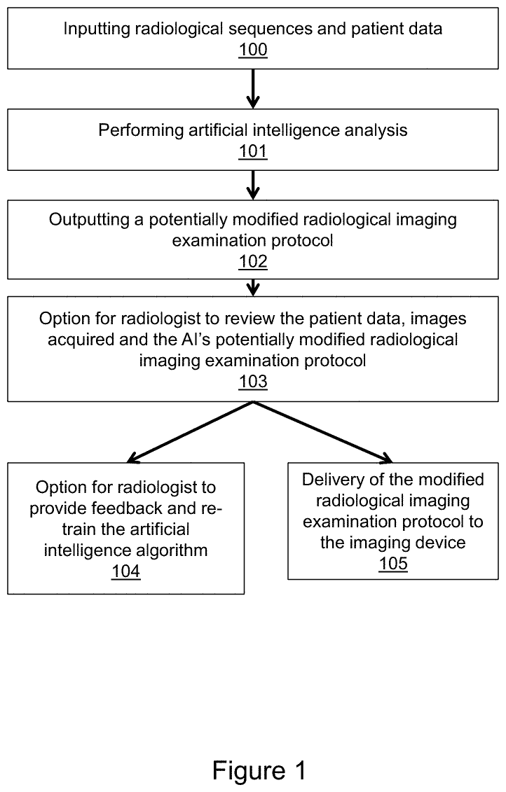

FIG. 1 illustrates a flow diagram for the method. Step 100 is to input radiological sequences and patient data. Step 101 is to perform artificial intelligence analysis. Step 102 is to output a potentially modified radiological imaging examination protocol. Step 103 is the option for radiologist to review the patient data, images acquired and the AI's potentially modified radiological imaging examination protocol. Step 104 is the option for the radiologist to provide feedback and re-train the artificial intelligence algorithm. Step 105 is to deliver the modified radiological imaging examination protocol to the imaging device.

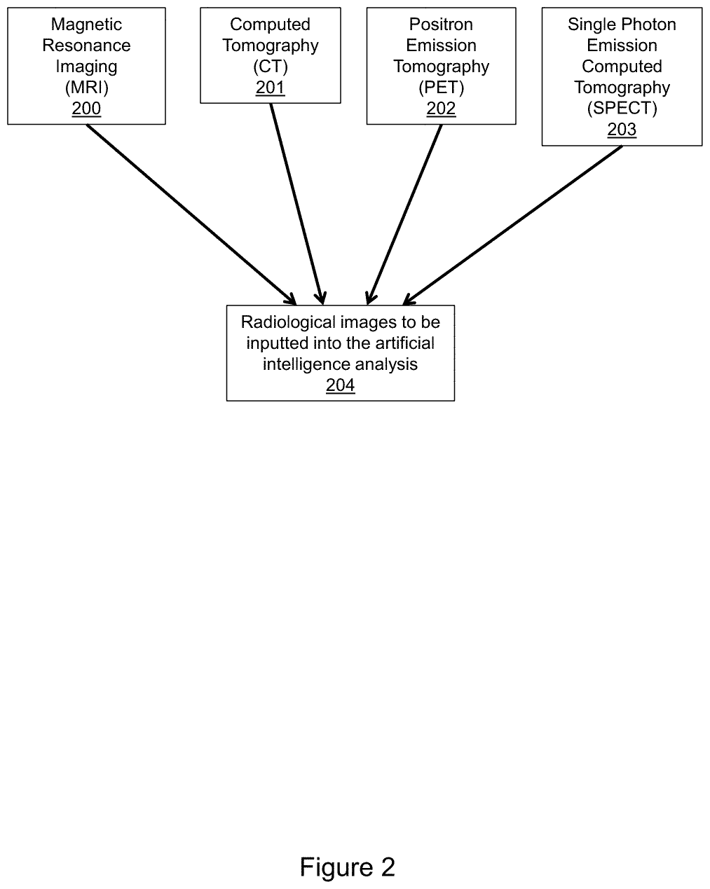

FIG. 2 illustrates inputted medical images includes magnetic resonance imaging (MRI) 200, computed tomography (CT) 201, positron emission tomography (PET) 202, single photon emission computed tomography (SPECT) 203. These radiological images would be inputted into the artificial intelligence system for analysis 204. An MRI examination typically consists of multiple sequences. For example, a routine brain MRI typically consists of a 3-plane single shot fast spin echo (SSFSE) localizer sequence, a sagittal T1-weighted sequence, an axial T2-weighted sequence, an axial gradient echo (GRE) sequence, an axial T2-weighted fluid-attenuated inversion recovery (FLAIR) sequence and an axial diffusion weighted imaging (DWI) sequence. The method would comprise sending each sequence performed from the MRI scanner to the computer system equipped with software to perform the artificial intelligence analysis as soon as the sequence is performed such that analysis can be performed and, if directed by the artificial intelligence algorithm, future sequences can be modified. During a CT scan, PET or SPECT scan, it is possible that the patient moves during some slices, but not during others. In this patent, the slices which were moved could be re-taken.

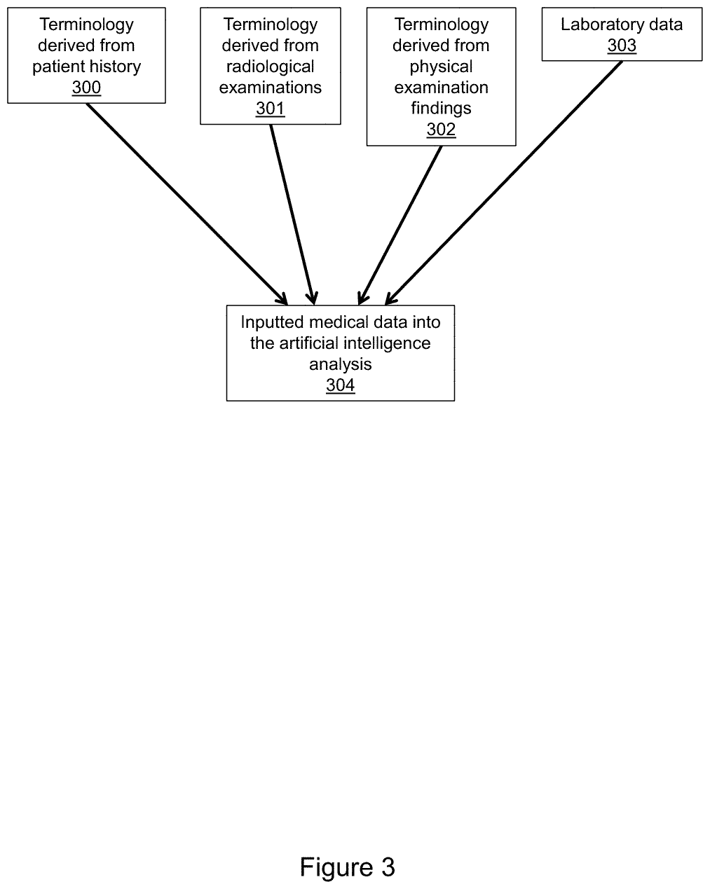

FIG. 3 illustrates examples of inputted medical data. Examples include terminology derived from patient history 300, terminology derived from radiological examinations 301, terminology derived from physical examination findings 302, and laboratory data 303. Some or all of the medical data is inputted to the artificial intelligence analysis 304.

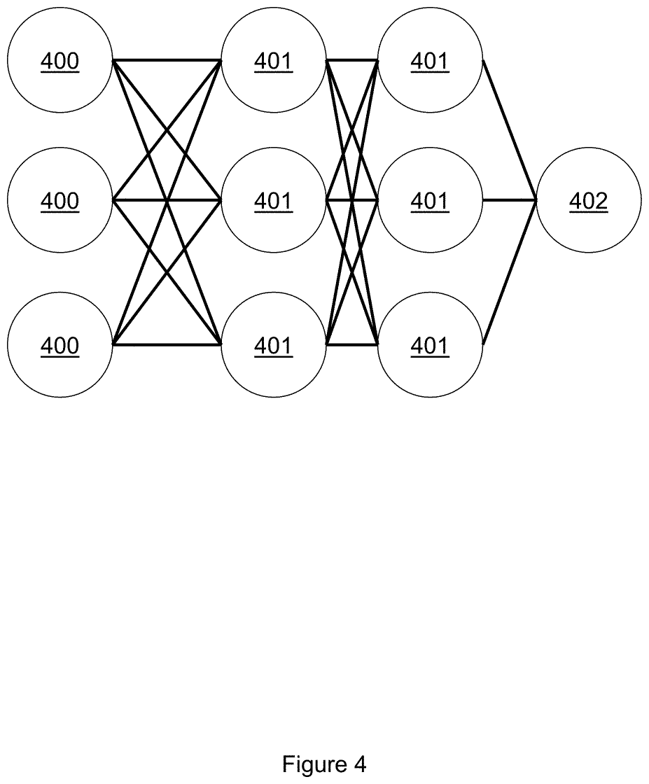

FIG. 4 illustrates sample artificial intelligence algorithms including deep artificial neural networks and other machine learning algorithms. Inputs 400, hidden layers 401 and an output 402 are shown. A variety of artificial intelligence algorithms are known, and it is assumed that the reader is a person of ordinary skill in the art with familiarity with artificial intelligence algorithms.

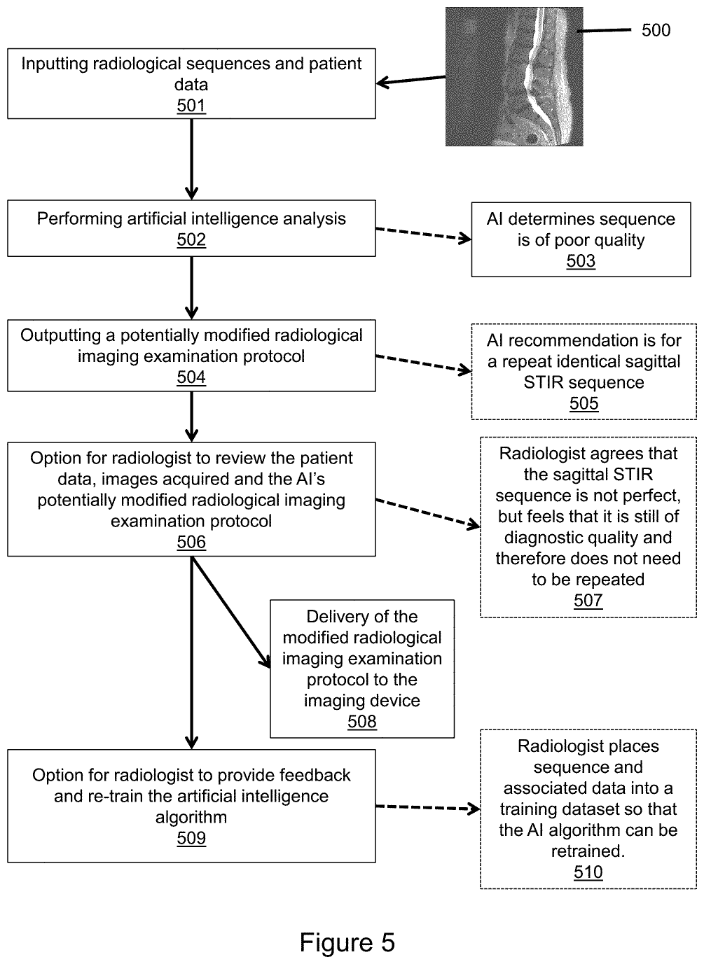

FIG. 5 illustrates performing analysis wherein the quality of the image(s) is assessed. A sagittal T2-STIR MRI sequence 500 is the inputted radiological sequence and patient data 501. AI analysis is performed 502. In this example, the AI may determine that the sequence is of poor quality 503. In this case, the AI system determines that the optimum step at this juncture is to modify the radiological imaging examination protocol and would send this output to the MRI scanner 504. AI recommendation is for a repeat identical STIR sequence 505. However, it should be noted that the sequence could be modified, such as a recommendation for a modified protocol (e.g., PROPELLAR sequence for motion correction). Various other parameters could be altered as determined by the AI system (e.g., flip angle, TR, TE or other MRI parameters). An option is for the radiologist to review the patient data, images acquired and the AI's potentially modified radiological imaging examination protocol 506. In this example, the radiologist agrees that the sagittal STIR sequence is not perfect, but feels that it is still of diagnostic quality and therefore does not need to be repeated 507. Thus, the radiologist could override the AI system. The modified radiological imaging examination protocol is delivered to the imaging device 508. An option for the radiologist is to provide feedback and re-train the artificial intelligence algorithm 509. The radiologist places sequence and associated data into a training dataset so that the AI algorithm can be retrained 510.

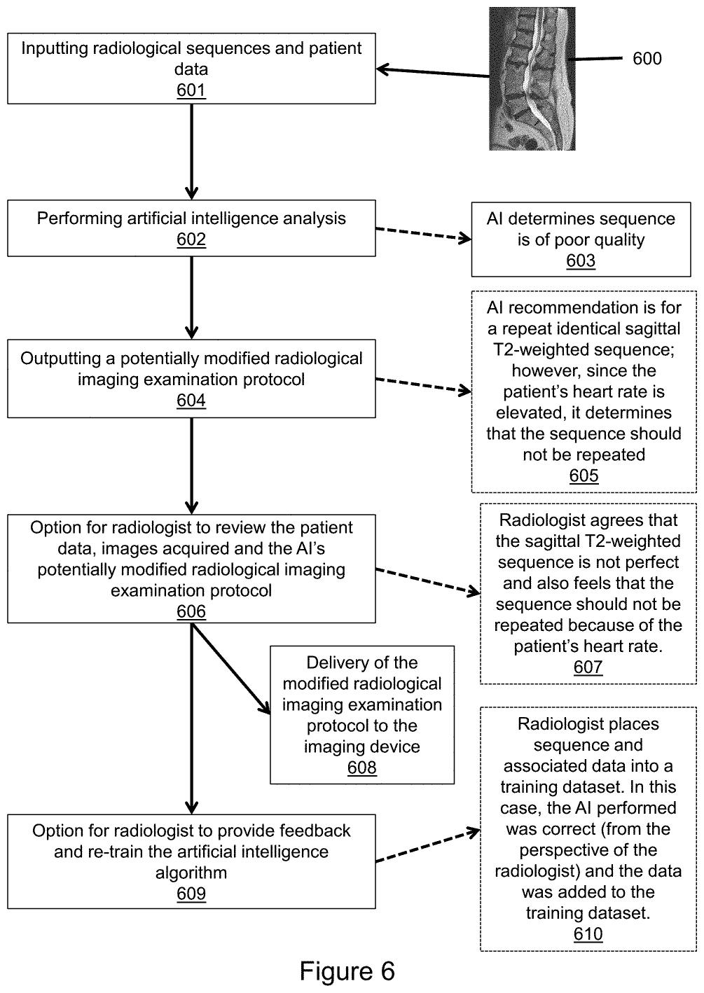

FIG. 6 illustrates performing analysis comprising assessment of the patient and determination of whether repeating the imaging examination is worthwhile. A T2-weighted sequence 600 is performed. The radiological sequences and patient data are inputted into the computer 601. The computer performs an artificial intelligence analysis process 602. The AI determines that the sequence is of poor quality 603. The AI outputs a potentially modified radiological imaging examination protocol 604. The AI recommendation is for a repeat identical sagittal T2-weighted sequence; however, since the patient's heart rate is elevated, it determines that the sequence should not be repeated 605. Thus, the AI system can weight in multiple factors in addition to the imaging findings to determine the best next step. The AI system would be programmed to explain the rationale of the decision to the user. An option at this point is for the radiologist to review the patient data, images acquired and the AI's potentially modified radiological imaging examination protocol 606. In this example, the radiologist agrees that the sagittal T2-weighted sequence is not perfect and also feels that the sequence should not be repeated because of the patient's heart rate 607. A modified radiological imaging examination protocol is delivered to the imaging device 608. An option at this point is for the radiologist to provide feedback and re-train the artificial intelligence algorithm 609. The radiologist places sequence and associated data into a training dataset. In this case, the AI performed was correct (from the perspective of the radiologist) and the data was added to the training dataset 610.

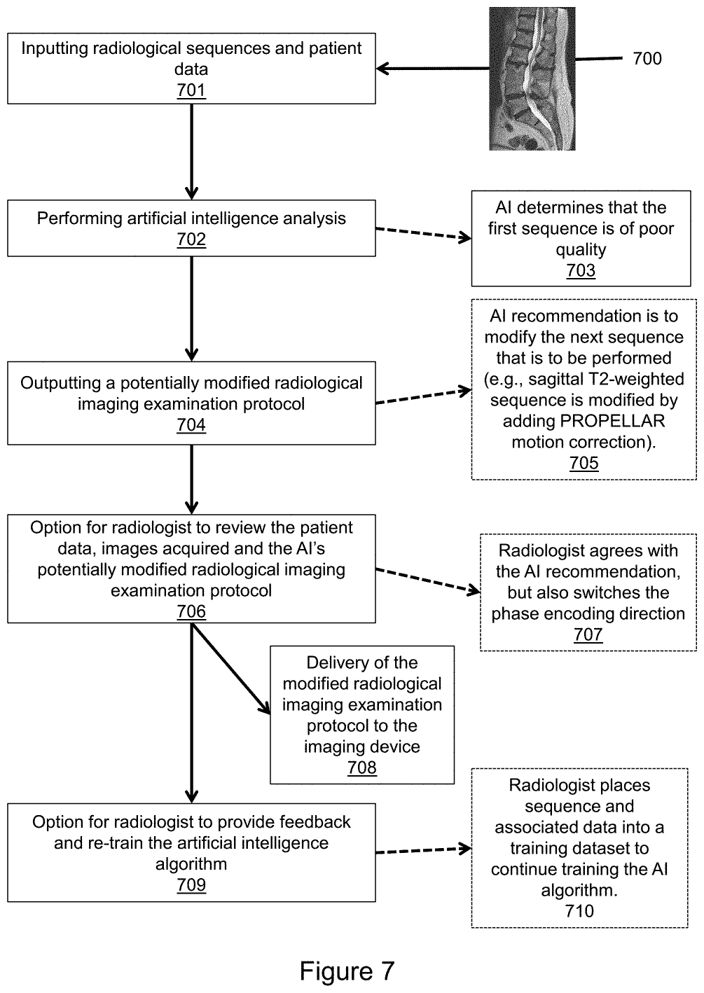

FIG. 7 illustrates AI altering the subsequent images scheduled to be performed. A sagittal T2-weighted sequence 700 is inputted 701 along with patient data into the AI system. A computer performs artificial intelligence analysis 702. The AI determines that the first sequence is of poor quality 703 and determines the reason for the poor quality. The AI system also outputs a potentially modified radiological imaging examination protocol 704. In this example, the AI recommendation is to modify the next sequence that is to be performed (e.g., sagittal T2-weighted sequence is modified by adding PROPELLAR motion correction) 705. There is an option for the radiologist to review the patient data, images acquired and the AI's potentially modified radiological imaging examination protocol 706. In this example, the radiologist agrees with the AI recommendation, but also switches the frequency encoding direction and the phase encoding direction 707. The modified radiological imaging examination protocol is delivered to the imaging device 708. There is an option for the radiologist to provide feedback and re-train the artificial intelligence algorithm 709. The radiologist places sequence and associated data into a training dataset to continue training the AI algorithm 710.

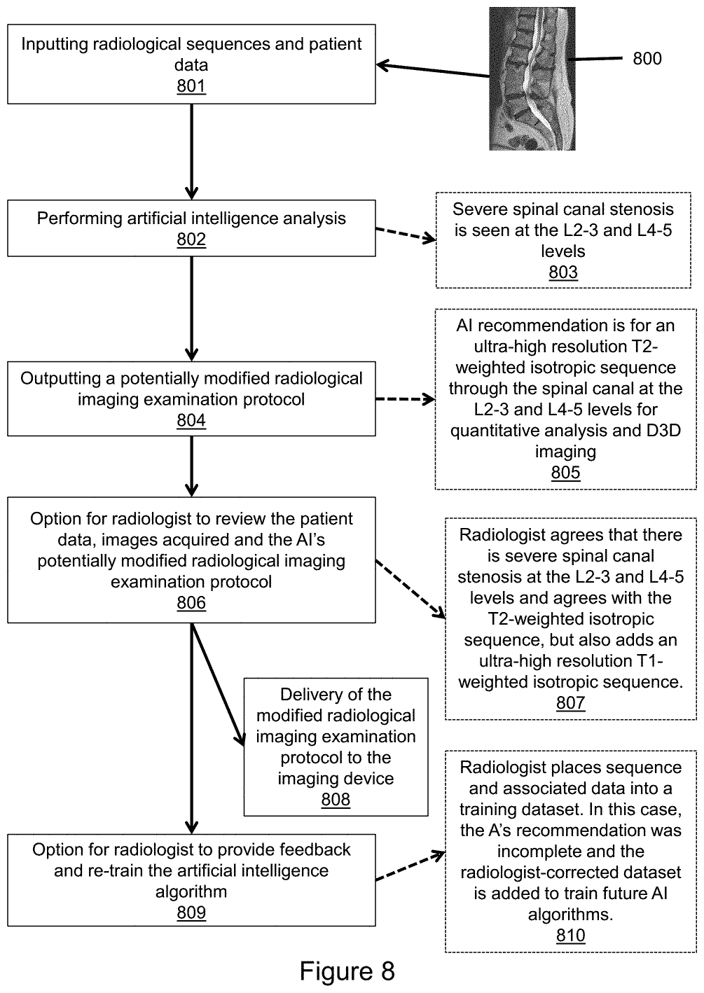

FIG. 8 illustrates AI performing analysis of pathology within the images and making recommendations for a modified radiological imaging examination protocol, which is then reviewed by a radiologist and the AI algorithm is subsequently retrained. A sagittal T2-weighted sequence 800 is inputted along with any other prior radiological sequences and other associated data (e.g., patient data, MRI schedule, etc.) 801. A computer performs AI analysis 802. The AI analysis determines that there is severe spinal canal stenosis at the L2-3 and L4-5 levels 803. The AI system outputs a potentially modified radiological imaging examination protocol 804. The AI recommendation is for an ultra-high resolution T2-weighted isotropic sequence through the spinal canal at the L2-3 and L4-5 levels for quantitative analysis and D3D imaging 805. An option is for the radiologist to review the patient data, images acquired and the AI's potentially modified radiological imaging examination protocol 806. The radiologist agrees that there is severe spinal canal stenosis at the L2-3 and L4-5 levels and agrees with the T2-weighted isotropic sequence, but also adds an ultra-high resolution T1-weighted isotropic sequence 807. The modified radiological imaging examination protocol is delivered to the imaging device 808. An option is for the radiologist to provide feedback and re-train the artificial intelligence algorithm 809. In this case the radiologist places sequence and associated data into a training dataset. In this case, the AI's recommendation was incomplete and the radiologist-corrected dataset is added to train future AI algorithms 810.

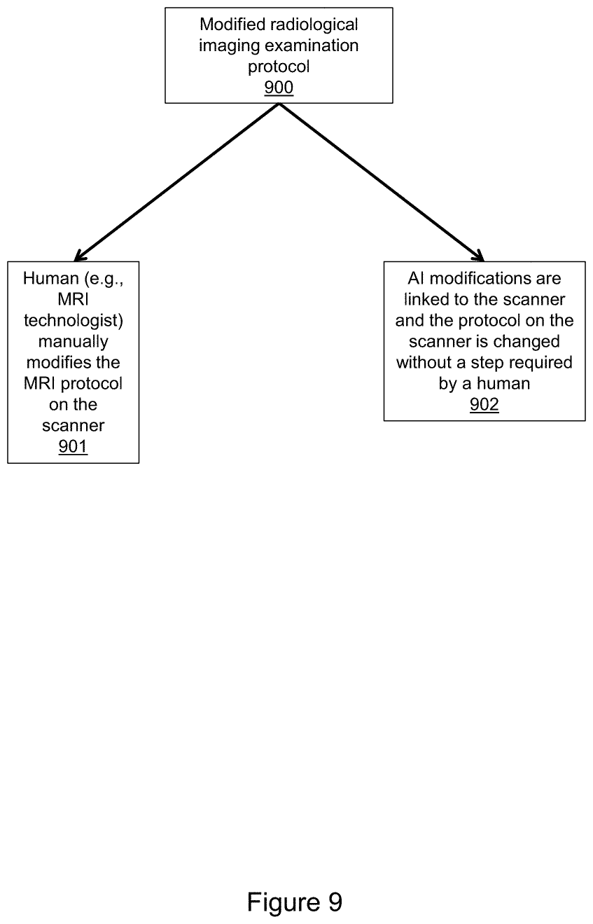

FIG. 9 illustrates methods to deliver the modified radiological examination protocol to the scanner. Modified radiological imaging examination protocol 900 is generated by the AI. A human (e.g., MRI technologist) manually modifies the MRI protocol on the scanner 901. Alternatively, AI modifications are linked to the scanner and the protocol on the scanner is changed without a step required by a human 902.

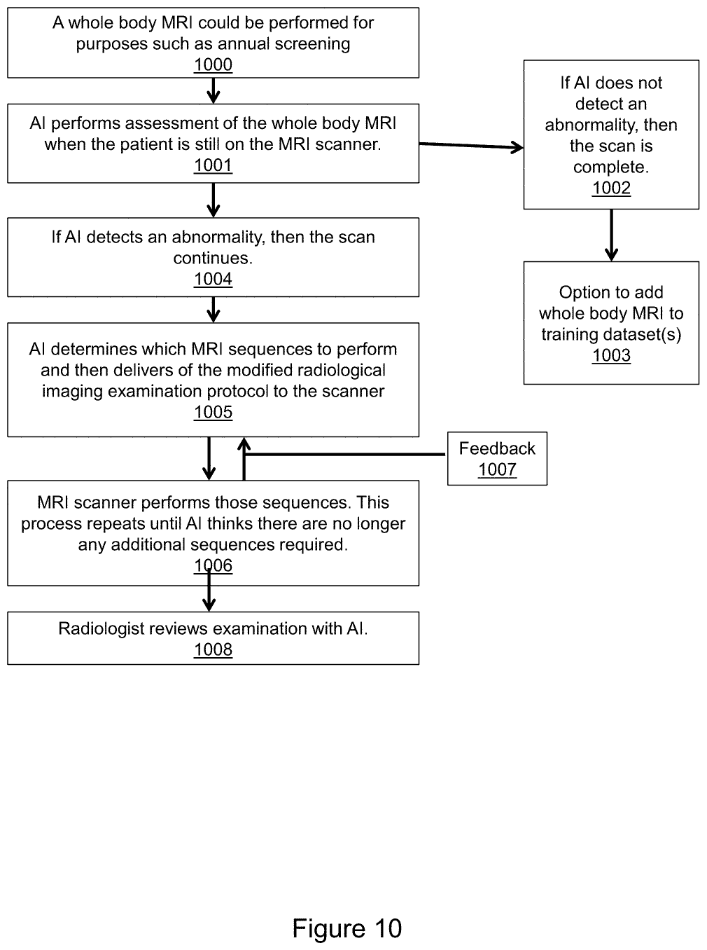

FIG. 10 illustrates an example use of this method for screening whole body MRI exams. The spatial resolution of such a scan could be low, such as 3 mm isotropic. Whole body analysis could be performed. Then, the AI could perform an assessment of the whole body MRI when the patient is still on the MRI scanner. If the AI doesn't detect an abnormality, then no additional sequences would be required. If desired, the volume could be sent to a database. If the AI did detect an abnormality, then the AI could communicate to the MRI scanner which sequences to perform. The MRI scanner would then perform those sequences. Then, the AI would review those sequences. This process would repeat until no additional sequences were performed. Finally, the radiologist would review the MRI examination with the AI. A whole body MRI could be performed for purposes such as annual screening 1000. The AI performs assessment of the whole body MRI when the patient is still on the MRI scanner 1001. If AI does not detect an abnormality, then the scan is complete 1002. An option is to add whole body MRI to training dataset(s) 1003. This can be performed with or without radiologist's second review. If AI detects an abnormality, then the scan continues 1004. The AI determines which MRI sequences to perform and then delivers of the modified radiological imaging examination protocol to the scanner 1005. The MRI scanner performs those sequences. This process repeats until AI thinks there are no longer any additional sequences required 1006. In addition, feedback 1007 will be performed based on the analytical results. Feedback would be at multiple aspects throughout this overall process. The radiologist reviews examination with AI 1008.

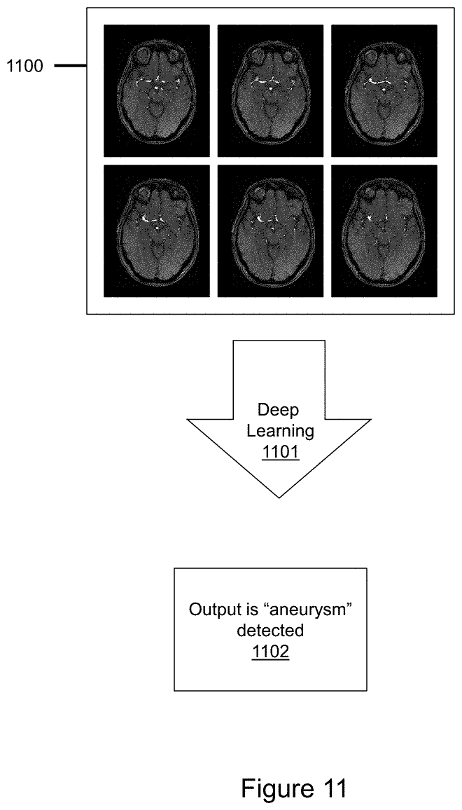

FIG. 11 illustrates a deep learning algorithm performed on source images to detect cerebral aneurysms, which has been performed in the prior art. An example of this is performed by Ueda et al. “Deep Learning for MR Angiography: Automated Detection of Cerebral Aneurysms” published online on 23 Oct. 2018 in Radiology. The article states that the algorithm was aimed “to detect cerebral aneurysms from TOF MR angiography source images.” In this figure, an example set of MRI source images 1100 are shown. A deep learning algorithm 1101 is shown. The output 1102 of such a system is whether or not an aneurysm is detected.

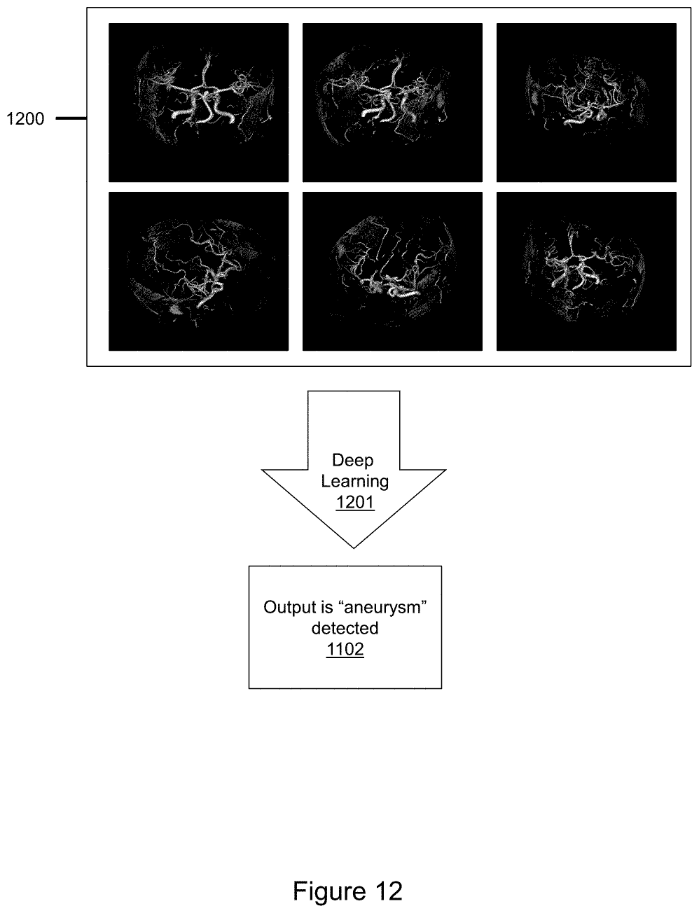

FIG. 12 illustrates a deep learning algorithm performed on processed images to detect cerebral aneurysms. In this figure, several processed images are created 1200 via filtering, segmentation and volume rendering. Pairs of stereoscopic images (i.e., left and right eye images) could also be performed. These processed images could be automated by a computer system and then inputted into a deep learning algorithm 1201 such that the output reveals the presence of absence of a brain aneurysm 1202. AI analysis on processed images has not been performed in the prior art. Inputting of processed images rather than raw data may be advantageous. In this example, the processed images that are inputted are volume rendered images that have been segmented and filtered. Specific types of processing prior to inputting into AI algorithms may yield more accurate classification. For example, for a basilar tip brain aneurysm, segmenting the vasculature, filtering (i.e., subtracting) the non-vasculature and inputting a view from the posterior angled toward the basilar tip might yield the highest accuracy. This type of image could be generated automatically while the individual is still on the MRI scanner and the AI system could direct the MRI scanner to perform any other images as needed to better characterize the basilar tip aneurysm. For an anterior communicating artery brain aneurysm, segmenting the vasculature, filtering (i.e., subtracting) the non-vasculature and inputting a view from the top (i.e., superior) looking down (i.e., inferior direction) and a second view from the bottom (i.e., inferior) looking up (i.e., superior direction) may yield the highest accuracy. Thus, different combinations of processed images inputted into an artificial intelligence algorithm may yield the highest possible classification accuracy and may surpass AI algorithms performed on source images as described in the prior art in FIG. 11. More generally, there are thousands of different pathologic diagnoses in the human body. A set of optimized processed images could be performed for each diagnosis and then sent to the AI system for classification (disease or no disease) and characterization (measurement thereof). This could be prioritized for different diagnoses, which are the most common and most dangerous first. For example, processed images that best depict intracranial hemorrhage, stroke, brain tumor, encephalitis and aneurysm could be performed first. Types of processing includes, but is not limited to, the following: windowing and leveling for a particular diagnosis; performing segmentation; performing filtering; performing volume rendering; and, performing D3D viewing in accordance with U.S. Pat. No. 8,384,771 A method and apparatus for three dimensional viewing of images. Then, more diagnoses can be added to the system. The majority of the diagnoses would likely have better detection by an AI system if a specific dedicated processed images (e.g., filtered, segmented image of the vasculature) is provided; however, some diagnoses would be better detected on source images. Therefore, a combination of source images and processed images would be used.

Specific examples have been presented to provide context and convey inventive concepts. The specific examples are not to be considered as limiting. A wide variety of modifications may be made without departing from the scope of the inventive concepts described herein. Moreover, the features, aspects, and implementations described herein may be combined in any technically possible way. Accordingly, modifications and combinations are within the scope of the following claims.

What is claimed is:

1. A method of modifying a radiological imaging examination protocol comprising:
inputting of medical images into a computer system;
performing artificial intelligence (AI) analysis of said medical images on said computer system;
outputting a modified radiological imaging examination protocol; and
delivering said modified radiological imaging examination protocol to the imaging device.

2. The method of claim 1 wherein inputting medical images comprises inputting at least one of: magnetic resonance imaging (MRI), computed tomography (CT), single photon emission computed tomography (SPECT) and positron emission tomography (PET).

3. The method of claim 1 comprising inputting medical images to be used by the AI system.

4. The method of claim 3 wherein inputting medical data comprises inputting at least one of the group of terminology derived from patient history, terminology derived from radiological examinations, terminology derived from physical examination findings, inputted medical data comprises laboratory data.

5. The method of claim 1 wherein performing analysis comprises utilizing artificial intelligence algorithms comprising at least one of deep learning, convolutional neural networks, recurrent neural networks, and reinforcement learning.

6. The method of claim 1 wherein performing analysis comprises performing assessment of the image(s).

7. The method of claim 6 wherein performing assessment of the image(s) comprises performing at least one of the group of assessment for image artifact, assessment for contrast resolution, assessment of spatial resolution, assessment for normal anatomy, assessment of pathology, performing measurements, anatomic positioning and anatomic posture.

8. The method of claim 7 comprising AI providing feedback to the scanner for possible re-imaging sequence.

9. The method of claim 1 comprising using image processing of medical images prior to performing the AI analysis.

10. The method of claim 9 wherein using image processing comprises at least one of the group of creating a 3D volumetric data set for the 2D, performing segmentation, performing filtering, performing measurements, and re-orienting volumetric data to optimize viewing geometry.

11. The method of claim 1 wherein performing analysis comprises performing assessment of the patient and determining whether repeating the imaging examination is worthwhile.

12. The method of claim 1 wherein outputting a modified radiological examination comprises adding new images to be performed comprising one of the group of CT images, MRI sequences, PET images, and SPECT images.

13. The method of claim 1 wherein outputting the modified radiological examination comprises prompting review by the radiologist to accept or reject the modifications to the radiological examination.

14. The method of claim 1 wherein outputting the modified radiological examination comprises at least one of the group of altering the subsequent images scheduled to be performed, removing subsequent images scheduled to be performed, and adding subsequent images.

15. The method of claim 1 wherein delivering the modified radiological imaging examination protocol to the imaging device comprises one of the group of a human entering modified protocol on the radiological scanner equipment and through an interface between the AI algorithm and the radiological scanner equipment.

16. The method of claim 1 comprising adding the inputted data and outputted data comprising the modified radiological imaging examination protocol to a training dataset for machine learning.

17. The method of claim 1 comprising performing whole body MRI screening examinations with AI driven focus areas for purposes including screening for pathology.

18. A method of performing artificial intelligence on radiological images comprising:
inputting of medical images into a computer system;
processing said images to optimize imaging of a particular pathology; and
performing artificial intelligence (AI) analysis of said processed medical images on said computer system for detection of said particular pathology.

19. The method of claim 18 wherein processing said images to optimize imaging of a particular pathology comprises one of the group of windowing and leveling, segmenting, filtering, volume rendering, and D3D viewing with images from multiple angles.

20. The method of claim 18 wherein processing said images to optimize imaging of a particular pathology comprises one of the group of utilizing a single processed image, utilizing multiple processed images, and utilizing a combination of processed images and source images.
